(19) United States
(12) Patent Application Publication
(10) Pub. No.: US 2013/0041033 A9
(43) Pub. Date: Feb. 14, 2013

(54) COMPOSITION COMPRISING VITAMIN U AS ACTIVE INGREDIENT FOR HEALING SKIN WOUND

(71) Applicants: Hyung Geun MIN, Seoul (KR); Won Serk KIM, Seoul (KR)

(72) Inventors: Hyung Geun MIN, Seoul (KR); Won Serk KIM, Seoul (KR)

(73) Assignee: CUSKIN CO., LTD., Seoul (KR)

(21) Appl. No.: 13/398,380

(22) Filed: Feb. 16, 2012

Related U.S. Application Data
(63) Continuation-in-part of application No. 13/152,681, filed on Jun. 3, 2011, now abandoned.
Application No. 13/152,681 is a division of application No. 12/736,189, filed on Sep. 17, 2010, now abandoned.

(30) Foreign Application Priority Data
Mar. 20, 2008 (KR) 10-2008-0025972

(51) Int. Cl. A61K 31/198; A61P 17/02

(52) U.S. Cl. 514/562

(57) ABSTRACT

Disclosed is a composition comprising vitamin U as an active ingredient, more precisely a composition for repairing the skin wound that contains vitamin U as an active ingredient. The composition demonstrates excellent wound healing and skin wound repairing effects.

### TECHNICAL FIELD

The present invention relates to a composition comprising vitamin U as an active ingredient, more precisely to a composition for healing skin wounds and repairing skin wound comprising vitamin U as an active ingredient.

### BACKGROUND ART OF THE INVENTION

Human skin is being aged as we are aged. There are many factors that affect skin aging such as UV, stress, disease, and other environmental factors. Fundamentally, as human beings are aged, fibroblasts and keratinocytes, major structural cells of skin, lose their functions and accordingly formation of skin collagen and dermal matrix is reduced. As a result, wound healing is retarded. And the enzyme that decomposes collagen (i.e. collagenase) is increased when the skin is exposed on UV for a long time, and then wound healing is retarded. Materials helpful for wound healing are exemplified by growth factors such as EGF and FGF, retinol, and vitamin C, etc. Cosmetics for reducing the effect of UV exposing containing such components are largely used.

Vitamin U is known to be a material effective in treating ulcer in the digestive system. Mucous membrane of the digestive system has a similar composition to that of the skin.

### SUMMARY OF THE INVENTION

As a result of the present inventors' studies in the utility of such composition comprising vitamin U that has not been well-known as an active ingredient so far, the present inventors have completed the present invention by finding out that the composition accelerates wound healing by a function of inhibiting collagenase.

It is an object of the present invention to provide a composition for healing the wound comprising vitamin U as an active ingredient.

It is another object of the present invention to provide a method for applying the composition comprising vitamin U on the skin for the purpose of healing the wound and repairing the skin wound.

Other objects and advantages of the present invention are more clearly understood by the following examples and claims.

The present invention relates to a composition comprising vitamin U as an active ingredient, more precisely to a composition for healing the wound and repairing the skin wound comprising vitamin U as an active ingredient. The composition of the present invention has an excellent effect on healing the wound and repairing the skin wound.

### BRIEF DESCRIPTION OF THE DRAWINGS

The above and other objects, features and advantages of the present invention will become apparent from the following description of preferred embodiments given in conjunction with the accompanying drawings, in which:

FIG. 1 is a set of graphs illustrating the cell growth of human fibroblasts treated with vitamin U at different concentrations analyzed by cell counting (left) and MTT assay (right).

FIG. 2 is a set of photographs illustrating the cell mobility of human fibroblasts treated with vitamin U at different concentrations over different time.

FIG. 3 is a graph illustrating the conversion of the cell mobility of human fibroblasts treated with vitamin U of FIG. 2 into numeric values.

FIG. 4 is a set of photographs illustrating the cell mobility of human keratinocytes treated with vitamin U at different concentrations over different time.

FIG. 5 is a graph illustrating the conversion of the cell mobility of human keratinocytes treated with vitamin U of FIG. 4 into numeric values.

FIG. 6 is a photograph illustrating the wound location of an atrichia mouse, the animal model for the experiment testing wound healing capacity of the composition comprising vitamin U of the present invention.

FIG. 7 is a set of photographs illustrating the wound healing effect in the atrichia mouse treated with the composition comprising vitamin U of the present invention at different concentrations over different time.

FIG. 8 is a graph illustrating the result of RT-PCR demonstrating the inhibition effect of the composition comprising vitamin U of the present invention on MMP-1 mRNA increased by UV irradiation.

### DETAILED DESCRIPTION OF THE INVENTION

Vitamin U is a nutrient rich in cabbage, broccoli, and seaweed, yet its physiological functions have not been well-known. Vitamin U is classified as methyl methionine sulfonium chloride in Merck index. Vitamin U is a low molecular weight fat-soluble material having the molecular weight of 199.70. The study of vitamin U has been started mainly in Eastern Europe. As known that vitamin U is effective in treating ulcer of the digestive system, it has been referred to as vitamin U, U being the initial letter of Ulcer. Other effects of vitamin U, such as anti-inflammation activity, improving activity of blood lipid, anti-anxiety effect, and inhibitory effect on cell membrane damage by stress in plants have been studied. Those effects could be brought mostly by oral administration.

The present inventors expected that vitamin U could be functioning for healing the skin wound and for cytoprotection similarly to healing ulcer or erosion of gastric mucosa when it was applied on the skin or infiltrated under the skin owing to its advantages of low-molecular weight and fat-solubility. From the observation that when vitamin U was administered, mucin secretion of gastric mucosa was increased in a rabbit, and ulcer of gastric mucosa was suppressed in a pig, it was expected that such anti-inflammatory effect and cytoprotection effect also could be observed on the skin that has a similar keratin structure to that of the mucous membrane.

As used herein, the term “vitamin U” can be extracted from various organs or parts of plants such as cabbage, broccoli, and seaweed (for example, leaves, flowers, roots, stems, branches, barks, fruits, and seeds, etc). A solvent usable for the extraction of vitamin U can be selected from the group consisting of (a) water, (b) C₁-C₄ anhydrous or hydrous lower alcohol (methanol, ethanol, propanol, and butanol, etc), (c) acetone, (d) ethyl acetate, (e) chloroform, (f) butyl acetate, and (g) 1,3-butylene glycol.

The extracts of the present invention include not only those extracted by using the said solvents but also other extracts obtained from the conventional purification processes. For example, fractions obtained from a variety of the additional purification processes such as the separation using a ultrafiltration membrane having a certain molecular weight cut-off value and the separation by chromatography (designed for the separation according to size, charge, hydrophobicity or affinity) are included in vitamin U of the present invention. In addition, other extracts obtained by the conventional extraction method such as supercritical extraction and ultrasonic extraction are also included.

Vitamin U of the present invention can be prepared in the form of powder by the additional process such as vacuum distillation, freeze drying or spray drying.

Either chemically synthesized or commercially-purchased vitamin U also can be used herein.

The composition of the present invention can contain other active ingredients than vitamin U. Specifically, the composition for wound healing and skin wound repairing can contain other wrinkle care components. However, the cosmetic composition of the invention is superior in its wound healing effect and wrinkle improving effect even with containing vitamin U alone as an active ingredient.

In a preferred embodiment of the present invention, the content of vitamin U in the composition is 0.00001-30%, preferably 0.0001-20%, more preferably 0.01-10.0%, and most preferably 0.1-10.0% by weight relative to the total weight of the composition.

If the content of vitamin U in the composition were less than the minimum, the wanted effect would not be expected. On the contrary, if the content were higher than the maximum, the effect would not be increased any longer and rather skin trouble might be caused and the stability of the formula could be in danger.

The composition of the present invention can include, in addition to the active ingredient, any conventional ingredients generally used in cosmetics, for example such additives and carriers as stabilizers, solubilizers, vitamins, pigments and flavors

The composition of the present invention can be formulated in any form that can be accepted in the art, which is exemplified by solution, suspension, emulsion, paste, gel, cream, lotion, powder, soap, surfactant-containing cleansing, oil, powdered foundation, emulsified foundation, wax foundation and spray, but not always limited thereto. More specifically, the composition of the present invention can be prepared in the form of soft lotion, nutrition lotion, nutrition cream, massage cream, essence, eye cream, cleansing cream, cleansing foam, cleansing water, pack, spray or powder.

In the case that the composition is formulated as paste, cream or gel, a suitable carrier can be selected from the group consisting of animal oil, vegetable oil, paraffin, starch, tragacanth, cellulose derivative, polyethylene glycol, silicon, bentonite, silica, talk and zinc oxide.

In the case that the composition is formulated as powder or spray, a suitable carrier can be selected from the group consisting of lactose, talc, silica, aluminum hydroxide, calcium silicate and polyamide powder, and in particular if the composition of the present invention is formulated as spray, a propellant such as chlorofluorohydrocarbon, propane/butane or dimethyl ether can be additionally included.

In the case that the composition is formulated as liquid or emulsion, a suitable carrier can be selected from the group consisting of solvent, solubilizer and emulsifier, which is exemplified by water, ethanol, isopropanol, ethyl carbonate, ethyl acetate, benzyl alcohol, benzyl benzoate, propylene glycol, 1,3-butyl glycol oil, glycerol aliphatic ester, polyethylene glycol and fatty acid ester of sorbitan.

In the case that the composition is formulated as suspension, a suitable carrier can be selected from the group consisting of liquid diluent such as water, ethanol or propylene glycol; suspending agent such as ethoxylated isostearyl alcohol, polyoxyethylene sorbitol ester and polyoxyethylene sorbitan ester; microcrystalline cellulose; aluminum methahydroxide; bentonite; agar; and tragacanth.

In the case that the composition is formulated as surfactant-containing cleansing, a suitable carrier can be selected from the group consisting of aliphatic alcohol sulfate, aliphatic alcohol ether sulfate, sulfosuccinic monoester, isethionate, imidazolium derivative, methyltaurate, sarcosinate, fatty acid amide ether sulfate, alkyl amidobetain, aliphatic alcohol, fatty acid glyceride, fatty acid diethanolamide, vegetable oil, lanolin derivative and ethoxylated glycerol fatty acid ester.

The present invention also provides a method of applying the composition of the present invention containing vitamin U on the skin to bring the effect of healing the wound and repairing the skin wound.

The makeup method of the present invention can include any method of applying a composition on human skin. That is, any method for applying a composition on human skin, which is well-known in the art, can pertain to the method of the present invention.

The composition of the present invention can be applied on the skin alone or in combination with other compositions, one time or multiple times. The composition having excellent cytoprotection effect of the present invention can be applied by the conventional method, and the number of times being applied depends on the skin condition or taste of a subject.

The composition of the present invention formulated in the form of soap, surfactant-containing cleansing or surfactant non-containing cleansing can be applied on the skin and then wiped out, removed or rinsed with water. Specifically, the soap of the invention can be prepared in the form of liquid soap, powder soap, solid soap and oil soap; the surfactant-containing cleansing formulation is exemplified by cleansing foam, cleansing water, cleansing towel and cleansing pack; and the examples of the surfactant non-containing cleansing are cleansing cream, cleansing lotion, cleansing water and cleansing gel, but not limited thereto.

The method of applying the composition comprising vitamin U of the present invention on human skin has proved to be very effective in healing the wound and repairing the skin wound.

Practical and presently preferred embodiments of the present invention are illustrative as shown in the following Examples.

However, it will be appreciated that those skilled in the art, on consideration of this disclosure, may make modifications and improvements within the spirit and scope of the present invention.

### EXAMPLES

Hereinafter, experimental examples are described.

The recovery process of wound is composed of the following three steps; inflammatory phase, collagen fiber phase or proliferative phase, and scar phase. Each step shows variations in characteristic cell compositions such as neutrophils, macrophages, fibroblasts and endothelial cells. These variations are important features to understand the degree of progress of wound recovery. The recovery procedure of wound is composed of contraction, epithelialization, collagen synthesis, angiogenesis, and granulation tissue generation. In the case of full-thickness excisional wound, contraction plays an important role. Meanwhile in the case of partial excisional wound where some of cutaneous Appendages remain, epithelialization is more important. The present inventors performed following experiments with the expectation that vitamin U would be a very helpful material for wound healing. Besides, the inventors also investigated if vitamin U had the preventive effect on a cause of skin-aging such as UV, etc.

### Experimental Example 1

### Proliferation of Human Fibroblasts

Using human dermal fibroblasts (hereinafter hDF), the present inventors investigated if vitamin U could accelerate the proliferation of fibroblasts in vitro. At this time, vitamin U was purchased from Fluka (DL-methionine methylsulfonium chloride; Fluka, Japan), and then diluted to 1 M. The basic culture medium for fibroblast was obtained from DMEM (Gibco, USA) with 10% FBS (Fetal Bovine Serum) and penicillin-streptomycin (antibiotic and antimicrobial agent). The medium used for the wound healing experiment was obtained from DMEM with 1% FBS. Such an amount of vitamin U was added that the final vitamin U concentration should be 10⁻² or 10⁻³ M. In the meantime, vitamin-free medium containing 1% FBS was used for the control.

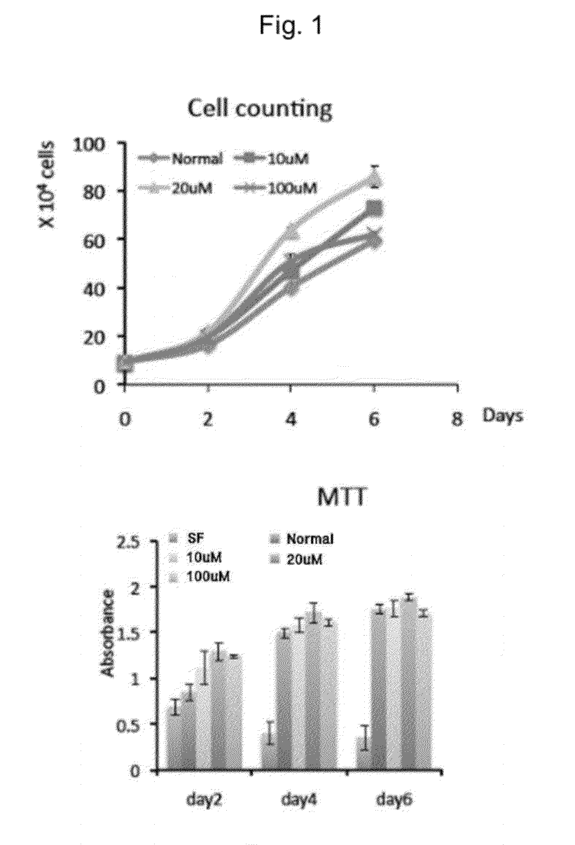

The cultured human fibroblasts were treated with different concentrations of vitamin U, followed by further culture. Cell proliferation was measured by cell counting and MTT assay. The results are presented as numeric values in the graph of FIG. 1.

### Experimental Example 2

### Increase of Mobility of Human Fibroblasts

Using human dermal fibroblasts (hereinafter hDF), the present inventors investigated if vitamin U could increase mobility of fibroblasts in vitro. At this time, vitamin U was purchased from Fluka (DL-methionine methylsulfonium chloride; Fluka, Japan), and then diluted to 1 M. The basic culture medium for fibroblast was obtained from DMEM (Gibco, USA) with 10% FBS (Fetal Bovine Serum) and penicillin-streptomycin (antibiotic and antimicrobial agent). The medium used for the wound healing experiment was obtained from DMEM with 1% FBS. Such an amount of vitamin U was added that the final vitamin U concentration should be 10⁻² or 10⁻³ M. In the meantime, vitamin-free medium containing 1% FBS was used for the control.

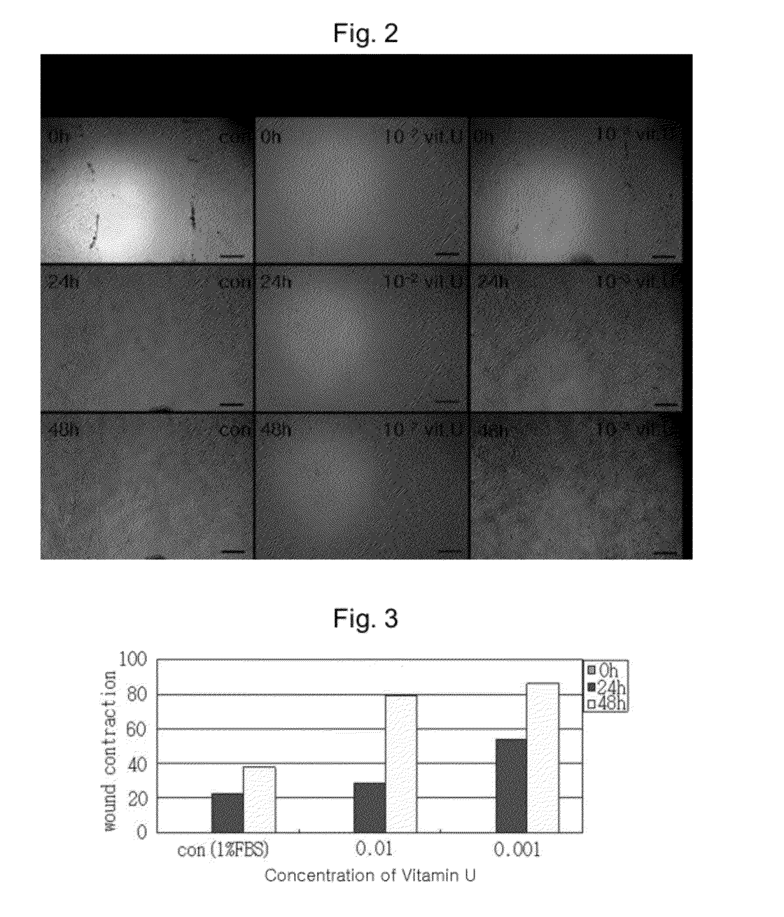

Human fibroblasts were cultured in a 6-well culture dish until the cells were grown at least 90% confluent. The cells were scratched out by using 2 mm micro tip. The fallen-off cells were washed out with PBS, followed by culture in another experimental medium. Then, it was observed by photograph how many cells migrate into the room between scratches. The results are presented as numeric values in FIG. 2 and FIG. 3.

As shown in FIG. 2 and FIG. 3, it could be confirmed that in the experimental group treated with vitamin U, the migration of fibroblasts was much more increased than in the control group not treated with vitamin.

### Experimental Example 3

### Increase of Mobility of Human Keratinocytes

The following experiment was performed to investigate if vitamin U could increase the movement of human epidermal keratinocytes (hereinafter hEK) in vitro. The basic culture medium was obtained from Defined Keratinocyte-SFM (Gibco, USA) with Defined Keratinocyte-SFM Growth supplement and growth factors. For the experiment, such an amount of 1/10 growth factor and vitamin U were added that the final concentrations of vitamin U should be 10⁻², 10⁻³, and 10⁻⁴M. In the meantime, vitamin-free medium containing 1/10 growth factor was used for the control.

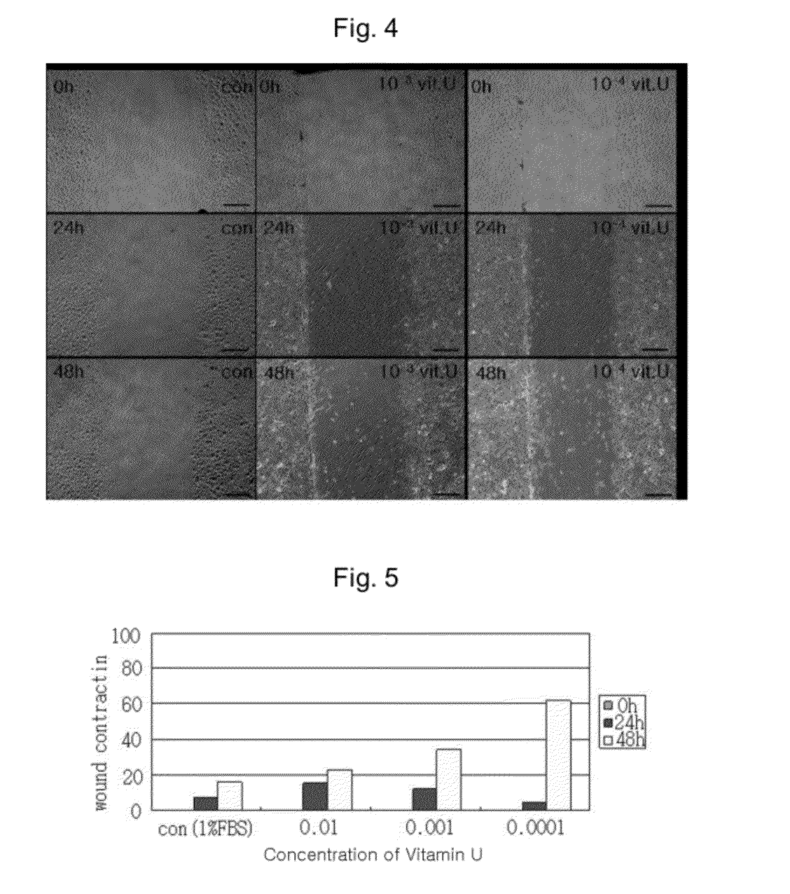

Human keratinocytes were cultured in a 6-well culture dish until the cells were grown at least 90% confluent. The cells were scratched out by using 2 mm micro tip. The fallen-off cells were washed out with PBS, followed by culture in another experimental medium. Then, it was observed by photograph how many cells migrate into the room between scratches (FIG. 4). The results are presented as numeric values in the graph of FIG. 5.

As shown in FIG. 4 and FIG. 5, it could be confirmed that in the experimental group treated with vitamin U, the migration of keratinocytes was much more increased than in the control group not treated with vitamin U.

Practical and presently preferred embodiments of the present invention are illustrative as shown in the following Preparative Examples.

However, it will be appreciated that those skilled in the art, on consideration of this disclosure, may make modifications and improvements within the spirit and scope of the present invention.

### Preparative Example 1

### Emollient Lotion (Skin Lotion)

Emollient lotion (skin lotion) containing vitamin U of the present invention was prepared according to the composition shown in Table 1.

TABLE 1

| Constituent | Content (weight %) |
|---|---|
| Vitamin U | 1 |
| Ethanol | 3 |
| Butylene glycol | 2 |
| Sodium hyaluronate | 1 |
| PEG-60 hydrogenated caster oil | 0.4 |
| Propylene glycol | 0.05 |
| Methylparaben | 0.15 |
| Chlorphenesin | 0.15 |
| Purified water | balance |
| Total | 100 |

### Preparative Example 2

### Nutritive Lotion (Milk Lotion)

Nutritive lotion (skin lotion) containing vitamin U of the present invention was prepared according to the composition shown in Table 2.

TABLE 2

| Constituent | Content (weight %) |
|---|---|
| Vitamin U | 1 |
| Butylene glycol | 5 |
| Cetyl caprylic | 3 |
| Squalane | 1 |
| Jojoba oil | 1 |
| Liquid paraffin | 1 |
| Cetyl alcohol | 0.4 |
| Stearic acid | 0.7 |
| Bees wax | 0.7 |
| Polysorbate 60 | 0.4 |
| Glyceryl stearate SE | 0.4 |
| Sorbate stearate | 0.3 |
| Disodium EDTA | 0.2 |
| Glyceryl stearate | 0.2 |
| PEG-100 stearate | 0.2 |
| Cabomer | 0.13 |
| Triethanolamine | 0.2 |
| Methylparaben | 0.2 |
| Propylparaben | 0.15 |
| Purified water | Balance |
| Total | 100 |

### Preparative Example 3

### Nutritive Cream

Nutritive cream containing vitamin U of the present invention was prepared according to the composition shown in Table 3.

TABLE 3

| | Exam- | Exam- | Exam- | Comparative |
|---|---|---|---|---|
| Constituent (weight %) | ple 1 | ple 2 | ple 3 | Example 1 |
| Vitamin U | 1 | 5 | 10 | — |
| Glycerine | 3 | 3 | 3 | 3 |
| Propylene glycol | 0.4 | 0.4 | 0.4 | 0.4 |
| Capric/caprylic | 3 | 3 | 3 | 3 |
| triglyceride | | | | |
| Cetearyl isononaniate | 3 | 3 | 3 | 3 |
| Butylene glycol | 2 | 2 | 2 | 2 |
| Cetearyl alcohol | 1.5 | 1.5 | 1.5 | 1.5 |
| Polyglyceryl-3 | 1 | 1 | 1 | 1 |
| methylglucose | | | | |
| distearate | | | | |
| Glyceryl polyethacrylate | 0.6 | 0.6 | 0.6 | 0.6 |
| Glyceryl stearate SE | 0.5 | 0.5 | 0.5 | 0.5 |
| Squalane | 0.5 | 0.5 | 0.5 | 0.5 |
| Glyceryl stearate | 0.4 | 0.4 | 0.4 | 0.4 |
| PEG-100 stearate | 0.4 | 0.4 | 0.4 | 0.4 |
| Polyacrylamide | 0.25 | 0.25 | 0.25 | 0.25 |
| Disodium EDTA | 0.02 | 0.02 | 0.02 | 0.02 |
| Methylparaben | 0.15 | 0.15 | 0.15 | 0.15 |
| Propylparaben | 0.12 | 0.12 | 0.12 | 0.12 |
| Purified water | Balance | Balance | Balance | Balance |
| Total | 100 | 100 | 100 | 100 |

### Preparative Example 4

### Massage Cream

Massage cream containing vitamin U of the present invention was prepared according to the composition shown in Table 4.

TABLE 4

| Constituent | Content (weight %) |
|---|---|
| Vitamin U | 1.0 |
| Glycerin | 4.0 |
| Vaseline | 3.5 |
| Triethanolamine | 0.5 |
| Liquid paraffin | 23.5 |
| Squalane | 3.0 |
| Bees wax | 2.1 |
| Tocopheryl acetate | 0.1 |
| Polysorbate 60 | 2.4 |
| Carboxylvinylpolymer | 1.0 |
| Sorbitan sesquioleate | 2.3 |
| Flavor | Traces |
| Antiseptic | Traces |
| Purified water | Balance |
| Total | 100 |

### Preparative Example 5

### Pack

Pack containing vitamin U of the present invention was prepared according to the composition shown in Table 5.

TABLE 5

| Constituent | Content (weight %) |
|---|---|
| Vitamin U | 1 |
| Polyvinylalcohol | 15 |
| Ethanol | 10 |
| Propylene glycol | 6 |
| Glycerin | 2 |
| Nonoxynol-12 | 0.5 |
| Methylparaben | 0.5 |
| Purified water | 65 |
| Total | 100 |

### Experimental Example 4

### Wound Healing Effect (In Vivo Animal Test)

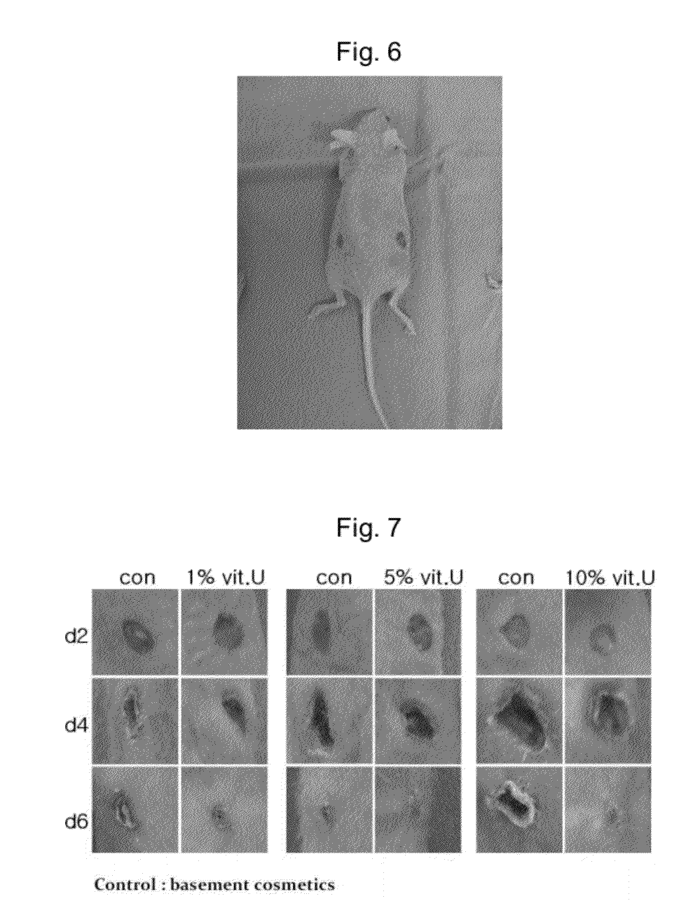

An experiment on Skin wound healing effect using the composition containing vitamin U of the present invention was carried out. Particularly, 6 atrichia mice were used as test animals. For the experiment, an anesthetic (zoletil-50: Rompun was diluted at the ratio of 2:1), punch biopsy (5 mm), the compositions of examples 1, 2, and 3 of preparative example 3 (the compositions contained vitamin U at the concentration of 1%, 5%, and 10%, respectively), and the composition of comparative example 1 that did not contained vitamin U were used. The atrichia mice were anesthetized with 40 mg/kg of the mixed anesthetic by intramuscular injection. Full-thickness excisional wound was made on both sides of the back skin of the anesthetized mouse by using punch biopsy (5 mm) (FIG. 6). On the left side of the wound, the composition of comparative example 1 was applied as the control, and on the right side of the wound, the compositions of Examples 1, 2, and 3 containing vitamin U at the concentrations of 1%, 5%, and 10%, respectively, were applied. Photographs were taken over the time. The results are shown in FIG. 7.

As shown in FIG. 7, the compositions of Examples 1, 2, and 3 containing vitamin U exhibited significantly higher wound healing effect than the composition of comparative example 1 that did not contain vitamin U. Particularly, the higher the concentration of vitamin U, the higher the wound healing effect went.

### Experimental Example 5

### Inhibition of Collagenase

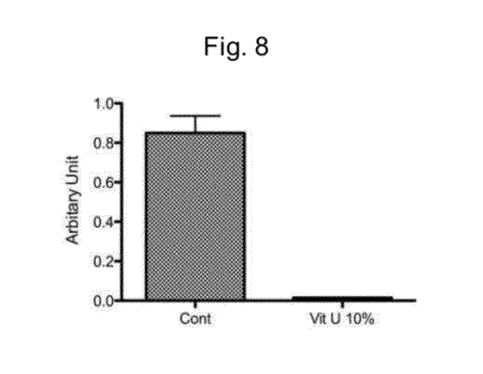

An experiment on Inhibitory effect on collagenase of the composition containing vitamin U of the present invention was carried out. The experiment was carried for 4 weeks with the Atrichia mice divided into two groups, each of which is composed of 10 mice. Vitamin U containing formula, prepared in cream with 10% vitamin U, was applied onto the back skin of the experimental group mouse every day. In the meantime, vitamin U-free ordinary formula was applied on the control. 4 weeks later, back skin was picked from both groups. RNA was extracted from the skin tissues, followed by real-time PCR to investigate the transcription level of collagenase mRNA (FIG. 8). As shown in FIG. 8, it could be known that the application of vitamin U brought significant inhibition of collagenase mRNA transcription. Western blotting was also performed with the same tissue samples to confirm the increase of collagen due to the decrease of collagenase (data not shown).

### Experimental Example 6

### Stability of Formulations

Stability of each composition of the present invention comprising vitamin U was tested. To investigate the stability of each composition, the cream of example 1 of preparative example 3 (containing vitamin U at the concentration of 1%) was put in opaque glass vessels, which were placed in incubators and maintained at the temperature of room temperature, 4° C., and 45° C., respectively, for 4 weeks, during which physical property (viscosity, pH) changes were observed. Discoloration by light was evaluated by comparing it with the standard color. Separation and discoloration were evaluated according to the following 6 grades.

<Evaluation Standard for Discoloration and Fragrance of the Product>

0: no changes observed, 1: minor changes observed,

2: moderate changes observed, 3: a little significant changes observed,

4: significant changes observed, 5: very significant changes observed

TABLE 6
Stability of formulation
Stability

| | | | Schedule | | | | | | |
|---|---|---|---|---|---|---|---|---|---|
| Item | Condition | | 0 Day | 3 Days | 7 Days | 14 Days | 21 Days | 28 Days | Test method |
| Viscosity | Room Temperature | Viscosity | 15000 | 15000 | 16000 | 16000 | 16000 | 16000 | Measured value, Viscometer |
| | (25° C.) | pH | 5.6 | 5.6 | 5.6 | 5.6 | 5.5 | 5.5 | Measured value, pH meter |
| | High Temperature | Viscosity | 15000 | 14000 | 14000 | 14000 | 14000 | 13000 | Viscometer, pH meter |
| | (45° C.) | pH | 5.6 | 5.5 | 5.5 | 5.5 | 5.5 | 5.5 | Measured by revert to 25° C. |
| | Cold Storage | Viscosity | 15000 | 15000 | 15000 | 15000 | 14000 | 14000 | Measured by revert to 25° C. |
| | (4° C.) | pH | 5.6 | 5.6 | 5.6 | 5.5 | 5.5 | 5.5 | Viscometer, pH meter |
| Color | Room Temperature | Color | 0 | 0 | 0 | 0 | 0 | 0 | Compared with standards |
| Fragrance | (25° C.) | Fragrance | 0 | 0 | 0 | 0 | 0 | 0 | |
| | High Temperature | Color | 0 | 0 | 0 | 0 | 0 | 1 | Compared with standards |
| | (45° C.) | Fragrance | 0 | 0 | 0 | 0 | 0 | 0 | |
| | Cold Storage | Color | 0 | 0 | 0 | 0 | 0 | 0 | Compared with standards |
| | (4° C.) | Fragrance | 0 | 0 | 0 | 0 | 0 | 0 | |
| | Day light | Color | 0 | 0 | 0 | 1 | 1 | 1 | Compared with standards |
| | (2 weeks) | Fragrance | 0 | 0 | 0 | 0 | 0 | 0 | |

* Standard preparation was sealed in an opaque glass vessel and stored in a dark room.
* Conditions for viscosity measurement: Brookfiele Helipath #6, 30 rmp, measured 1 minute layer (unit: cps)

Those skilled in the art will appreciate that the conceptions and specific embodiments disclosed in the foregoing description may be readily utilized as a basis for modifying or designing other embodiments for carrying out the same purposes of the present invention. Those skilled in the art will also appreciate that such equivalent embodiments do not depart from the spirit and scope of the invention as set forth in the appended claims.

What is claimed is:

1. A method of healing skin wound and inhibiting collagenase, the method comprising applying an effective amount of a composition comprising vitamin U as an active ingredient to a skin wound on human skin.

2. The method according to claim 1, wherein the vitamin U is included at a concentration of 0.00001-30.0% (w/w) by the total weight of the composition.

3. The method according to claim 1, wherein the composition is formulated as a form selected from the group consisting of solutions, suspensions, emulsions, pastes, gels, creams, lotions, powders, soaps, surfactant-containing cleaners, oils, powdered foundations, emulsified foundations, wax foundations, and sprays.

4. A method of using a composition comprising vitamin U as an active ingredient to heal skin wound and inhibit collagenase, the method comprising the step of applying an effective amount of the composition to a skin wound on human skin.

5. The method according to claim 4, wherein the vitamin U is included at a concentration of 0.00001-30.0% (w/w) by the total weight of the composition.

6. The method according to claim 4, wherein the composition is formulated as a form selected from the group consisting of solutions, suspensions, emulsions, pastes, gels, creams, lotions, powders, soaps, surfactant-containing cleansers, oils, powdered foundations, emulsified foundations, wax foundations, and sprays.
